(19) United States
(12) Patent Application Publication
(10) Pub. No.: US 2026/0348901 A1
(43) Pub. Date: Nov. 5, 2026

(54) METHOD FOR FUNCTIONALIZING A SURFACE WITH A BIOMOLECULE IN A LAYER HAVING ANTIFOULING PROPERTIES

(71) Applicants: IMEC VZW, Leuven (BE); Katholieke Universiteit Leuven, Leuven (BE); MOLECULAR PLASMA GROUP BV, Leuven (BE)

(72) Inventors: Tijs Dekoster, Tildonk (BE); Karolien Jans, Tessenderlo (BE); Rita Vos, Baal (BE); Willem Van Roy, Bierbeek (BE); Annelies Delabie, Bierbeek (BE)

(21) Appl. No.: 19/662,531

(22) Filed: Apr. 29, 2026

(30) Foreign Application Priority Data
Apr. 30, 2025 (EP) 25173637.7

(51) Int. Cl. G01N 27/327; C23C 16/448; C23C 16/513

(52) U.S. Cl. CPC G01N 27/3275; C23C 16/4486; C23C 16/513

(57) ABSTRACT

The present disclosure relates to functionalizing a surface of a biosensor with a biomolecule. The disclosure provides a method for codepositing the biomolecule and an organic precursor onto the surface, and provides a biosensor with a functionalized surface. The method includes a step of codepositing the biomolecule and an organic precursor onto the surface using atmospheric pressure plasma deposition. To obtain a stable layer with antifouling properties on the surface, the organic precursor includes a polymerizable group and an ethylene oxide.

### CROSS-REFERENCE TO RELATED APPLICATIONS

The present application is a non-provisional patent application claiming priority to European Patent Application No. EP 25173637.7, filed Apr. 30, 2025, the contents of which are hereby incorporated by reference.

### FIELD OF THE DISCLOSURE

The present disclosure relates to functionalizing a surface of a biosensor with a biomolecule. To this end, the disclosure provides a method for codepositing the biomolecule and an organic precursor onto the surface. The method leads to the incorporation of the biomolecule into a stable biocompatible polymer layer with antifouling properties.

### BACKGROUND

Biosensors play an increasingly important role in our daily life, with applications ranging from measuring the glucose level in the blood to detecting infectious diseases. Crucial for most biosensors is the functionalization of a surface with a biomolecule of interest using a linker layer. To deposit the linker layer, new technologies are investigated, such as atmospheric pressure plasma deposition (APPD). This technology potentially allows the biomolecule to be deposited in a more cost-effective and scalable way.

During APPD, a plasma of a carrier gas, such as helium (He), argon (Ar), or nitrogen (N₂), is generated in between two electrodes, and is blown out of the region in between the electrodes by a flow of the carrier gas. A precursor is injected into the afterglow of the plasma, to limit fragmentation and to allow the deposition of fragile biomolecules and organic precursors.

A first example approach of using APPD to deposit a biomolecule onto a substrate surface, which will provide the surface, involves a two-step process. In this process, an organic linker layer is deposited in a first step, and the biomolecule is deposited on the linker layer in a second step. A two-step process is, however, not ideal. A second example approach comprises a one-step process, where an aerosol of a biomolecule and vapor of a hydrocarbon precursor (such as ethylene or acetylene) are copolymerized by the plasma during APPD onto a surface. However, this approach leads to a carbon rich linker layer with unfavorable properties for both the survival of the biomolecule and the fouling of the surface.

### SUMMARY

In view of the above, an objective of the disclosure is to provide a method for functionalizing a surface of a biosensor with a biomolecule, wherein the method overcomes the issues of the conventional approaches that use APPD. For instance, an objective is to enable the functionalization in a one-step APPD process. Another objective is to provide for antifouling properties, to prevent the layer on the surface from fouling, for example, by proteins or cells. Another objective is to achieve sufficient cross-linking to avoid dissolution of the layer in aqueous solutions and prevent leaching of the biomolecules. Another objective is to retain the bioactivity and stability of the biomolecules.

These and other objectives may be achieved by example embodiments of this disclosure, which are described in the independent claims. Further example implementations are described in the dependent claims.

A first aspect of this disclosure provides a method for functionalizing a surface of a biosensor with a biomolecule, the method comprising: codepositing the biomolecule and an organic precursor onto the surface using APPD; wherein the organic precursor comprises a polymerizable group and an ethylene oxide functional group.

The use of the APPD technology in the method of the first aspect enables co-injection of the biomolecule and the organic precursor, can be performed at atmospheric pressure and with a mild plasma, is solvent free, and is suitable for the treatment of large surface areas.

The method of the first aspect enables a one-step process, and produces a film on the surface, which shows good cross-linking to prevent dissolution in aqueous solutions, has antifouling properties—due to the selection of the organic precursor—that prevent fouling by proteins or cells, and immobilizes the biomolecules on the senor surface while retaining their bioactivity and stability.

A biomolecule is a chemical compound naturally found in living organisms, examples including proteins, nucleic acids, carbohydrates, and lipids. Notably, the “biomolecule” in this case means a type of biomolecule. That is, the term “biomolecule” may not strictly refer to a single “instance” of the biomolecule, i.e. an individual molecule, but to a class, and many copies of that biomolecule can be deposited.

During the deposition, the surface may be a surface of a substrate. The substrate with the functionalized surface may subsequently be used to process the complete biosensor, for example, by processing a transducer of the biosensor. The “surface of a biosensor” in this disclosure also relates to such situations.

A polymerizable group may be a reactive moiety, like vinyl or acrylate, that enables monomers to bond and form polymers through polymerization.

A biosensor is a combination of a functionalized surface including a bio-receptor—e.g. the surface functionalized with the biomolecule- and a transducer. The bio-receptor (or short receptor) has sensitivity and selectivity to a target, e.g., an analyte that may bind to the bio-receptor. A transducer of a biosensor is an electrical or optical component that converts the biochemical interaction of e.g. the target with the bio-receptor into an electronic signal.

In an example implementation of the method, the polymerizable group of the organic precursor comprises a vinyl, or an acrylate, or a methacrylate, or an epoxy, or a silane.

In an example implementation of the method, the organic precursor comprises at least two polymerizable groups.

In an example implementation of the method, the organic precursor comprises at least two vinyl, acrylate, methacrylate, or epoxy, silane groups or combinations thereof.

In an example implementation of the method, the organic precursor comprises two acrylate or two methacrylate or two vinyl groups.

In an example implementation of the method, the organic precursor further comprises a spacer, the spacer comprising an alkane, or an ether, or a ketone, or an ester, or an amide.

In an example implementation of the method, the spacer of the organic precursor comprises an ether, such as a glycol ether.

In an example implementation of the method, the spacer of the organic precursor comprises at least 2, such as at least 3, or at least 9 repeating units of alkane, or an ether, or a ketone, or an ester, or an amide, such as a glycol ether.

The spacer may be used to increase the anti-fouling properties of the functionalized surface. The ratio of the reactive group over length also determines the density and/or porosity of the layer (or film) on the surface, and additionally enables crosslinking of the biomolecule into the formed polymer.

In an example implementation of the method, the spacer and the polymerizable group of the organic precursor are not the same.

In an example implementation of the method, the organic precursor is selected from the group consisting of ethylene glycol dimethacrylate (EGDMA), di(ethylene glycol) dimethacrylate (2EGDMA), tri(ethylene glycol) dimethacrylate (3EGDMA), polyethylene glycol dimethacrylate (PEGDMA), ethylene glycol divinyl ether, di(ethylene glycol) divinyl ether, tri(ethylene glycol) divinyl ether, and polyethylene glycol divinyl ether.

In an example implementation of the method, the polyethylene glycol dimethacrylate has a molecular weight of at least 330 g/mol, such as at least 600 g/mol.

In an example implementation of the method, the polyethylene glycol dimethacrylate has a molecular weight of less than or equal to 1000 g/mol, for example, less than or equal to 800 g/mol.

In an example implementation of the method, the polyethylene glycol dimethacrylate has a molecular weight of at least 400 g/mol and less than or equal to 1000 g/mol, such as at least 600 g/mol and less than or equal to 800 g/mol.

In an example implementation of the method, the polyethylene glycol divinyl ether has a molecular weight of at least 100 g/mol, for example at least 150 g/mol.

In an example implementation of the method, the polyethylene glycol divinyl ether has a molecular weight of less than or equal to 300 g/mol, such as less than or equal to 250 g/mol.

In an example implementation of the method the polyethylene glycol divinyl ether has a molecular weight of at least 100 g/mol and less than or equal to 300 g/mol, for example at least 150 and less than or equal to 250 g/mol.

In an example implementation of the method, the biomolecule is one of an enzyme, an antibody, a nanobody, an aptamer, DNA, RNA, and a peptide.

In an example implementation of the method, the biomolecule is glucose oxidase.

In an example implementation of the method, the organic precursor is di(ethylene glycol) divinyl ether or poly(ethylene glycol) dimethacrylate, and/or the biomolecule is glucose oxidase.

In an example implementation of the method, the biomolecule and the organic precursor are codeposited assisted by a plasma afterglow during the APPD or are codeposited and then treated with the plasma afterglow.

In an example implementation of the method, during the APPD, the organic precursor is injected as vapor or an aerosol into the plasma afterglow, and the biomolecule is injected as an aerosol into the plasma afterglow.

A suitable solvent for the biomolecule aerosol may be water.

In an example implementation of the method, the biomolecule and the organic precursor are dissolved in water, and during the APPD the water comprising the dissolved organic precursor and biomolecule is injected into the plasma afterglow.

For example, the biomolecule and the organic precursor may be dissolved in water e.g., 25 mg/mL glucose oxidase in water, containing also 10V % poly(ethylene glycol) dimethacrylate.

In an example implementation of the method, the biomolecule and the organic precursor are injected from a common container or from separate containers into the plasma afterglow.

In an example implementation of the method, a water aerosol is injected together with the organic precursor and the biomolecule into the plasma afterglow during the APPD.

In an example implementation of the method, argon or helium or nitrogen is used as plasma gas for the APPD.

In an example implementation of the method, the codepositing of the organic precursor and the biomolecule forms a film on the surface.

In an example implementation of the method, the codepositing of the organic precursor and the biomolecule is performed in the presence of an inert gas flux, such as an argon or nitrogen flux.

The presence of an inert gas flux during codeposition removes ambient air from the deposition area and thereby reduces the oxygen content during codeposition.

The film is stable, for instance against aqueous dissolution, and has antifouling properties.

A second aspect of this disclosure provides a biosensor comprising: a surface on which a biomolecule is immobilized; wherein a film comprising the biomolecule and an organic precursor is formed on the surface; and wherein the organic precursor comprises a polymerizable group and an ethylene oxide functional group.

In an example implementation of the biosensor, the film comprises a matrix formed by the ethylene oxide containing organic precursor and comprises the biomolecule dispersed in the matrix; and optionally the biomolecule is distributed in the film or on top of the film.

In example embodiments, the biosensor may be have technological advantages in that the layer on the surface is stable, retains the bioactivity of the biomolecule(s), and has antifouling properties.

### BRIEF DESCRIPTION OF THE FIGURES

The above, as well as additional, features will be better understood through the following illustrative and non-limiting detailed description of example embodiments, with reference to the appended drawings.

FIG. 1 schematically illustrates a method for functionalizing a surface of a biosensor with a biomolecule, according to example embodiments.

FIG. 2 illustrates a device for performing APPD, the device being configured to codeposit the biomolecule and the organic precursor from a common container, according to example embodiments.

FIG. 3 illustrates a device for performing APPD, the device being configured to codeposit the biomolecule and the organic precursor from separate containers, according to example embodiments.

FIG. 4A shows a biosensor with a functionalized surface according to example embodiments.

FIG. 4B shows an example of an organic precursor, according to example embodiments.

FIG. 4C shows an example of an organic precursor, according to example embodiments.

FIG. 5A shows an image of a functionalized surface comprising an organic precursor matrix and biomolecule, according to example embodiments.

FIG. 5B shows an image of a functionalized surface comprising an organic precursor matrix and biomolecule, according to example embodiments.

FIG. 6A shows dose-response curves for glucose in PBS of GOx/PEO films deposited without water co-injection, according to example embodiments.

FIG. 6B shows dose-response curves for glucose in PBS of GOx/PEO films deposited with water co-injection, according to example embodiments.

FIG. 7 shows a table with the apparent K_{M}, Jₘₐₓ, and sensitivity for the films deposited at different precursor mass flows and with and without water co-injection, according to example embodiments.

FIG. 8A shows dose-response curves for glucose in PBS after incubation in cell culture medium with 10 vol % FBS for 12 h and 36 h incubation, according to example embodiments.

FIG. 8B shows dose-response curve for glucose in PBS for a film deposited at 106 mg/min mass flow with water co-injection flow after storing the film in a dry environment for 1 and 4 weeks, according to example embodiments.

FIG. 9A shows integrated intensity of the amide I band in the transmission FT-IR spectra, according to example embodiments.

FIG. 9B shows transmission FT-IR spectra of films deposited from a 25 mg/mL GOx 10 vol % PEGDMA solution, according to example embodiments.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

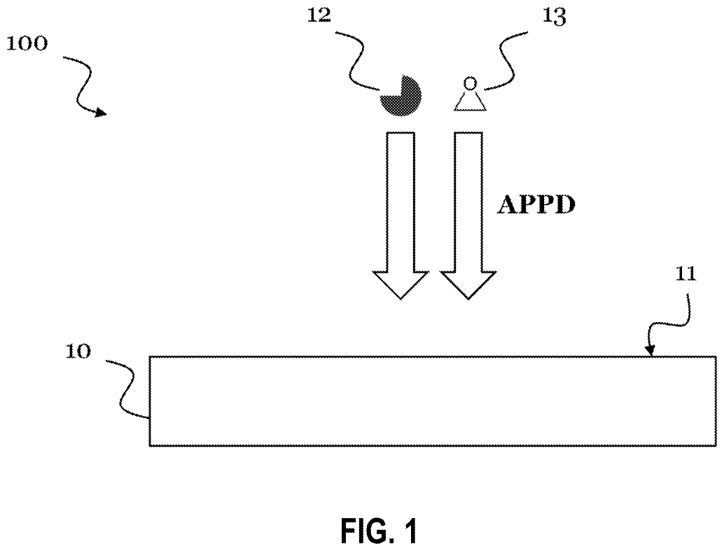

FIG. 1 illustrates a method 100 for functionalizing a surface 11 of a biosensor 10 with a biomolecule 12. The biomolecule 12 may be any one of an enzyme, an antibody, a nanobody, an aptamer, DNA, RNA, and a peptide. For example, the biomolecule 12 may be glucose oxidase. The biosensor 10 may have a solid support structure, such as a metal, oxide, or carbon electrode formed by a substrate, a surface 11 of which may be applied with the biomolecule 12. During the APPD, the biosensor 10 may yet be in a pre-processed state, and the method 100 may be used to functionalize a substrate surface, which will become the surface 11 of the biosensor 10 after processing the biosensor 10 is complete.

In an example embodiment, the biosensor 10 may be a glucose biosensor, where glucose oxidase enzymes may be functionalized on the electrode to detect blood sugar levels. In another example embodiment, the biosensor 10 may be an immunosensor, which uses antibodies on the sensor surface 11 to specifically bind target antigens for disease diagnostics.

The method 100 comprises codepositing the biomolecule 12 and an organic precursor 13 onto the surface 11 using APPD. APPD is a deposition technique that uses a plasma at ambient pressure to deposit thin films on surfaces. APPD enables precise functionalization of materials by introducing reactive species that facilitate polymerization or grafting of biomolecules, making it useful for biosensor surface modification

The organic precursor 13 used in this disclosure comprises a polymerizable group and an ethylene oxide. The polymerizable group of the organic precursor 13 may comprise a vinyl, or an acrylate, or a methacrylate, or an epoxy, or a silane. For instance, the organic precursor 13 may be di(ethylene glycol) divinyl ether or may be poly(ethylene glycol) dimethacrylate. The organic precursor 13 may further comprise a spacer, for instance, an alkane, or an ether, or a ketone, ester, amide.

The organic precursor 13 used in this disclosure may be purposefully selected, according to the following:
(1) The polymerizable group (e.g., vinyl, acrylate, methacrylate, epoxy, silane . . . ) may be used to provide cross-linking.
(2) The ethylene oxide ((CH₂—CH₂—O)ₙ, n>=2) may be used to provide the antifouling properties.
(3) The optional spacer (alkane, ether, ketone . . . ) may be used to support the functionality and bioactivity of the biomolecules on the surface 11.

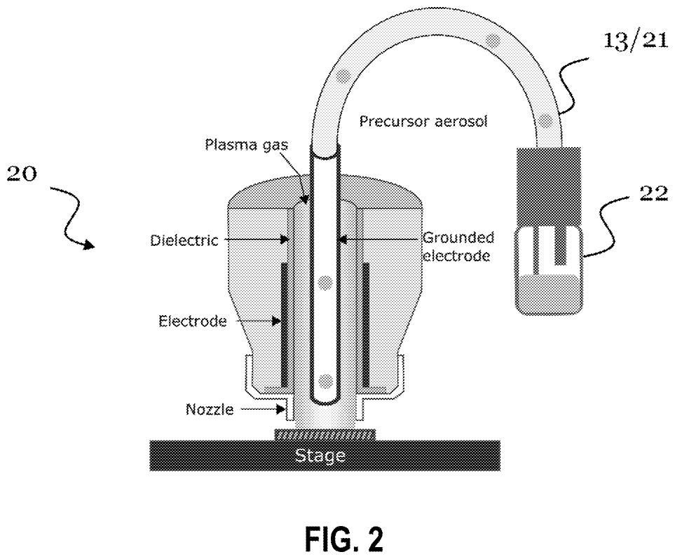

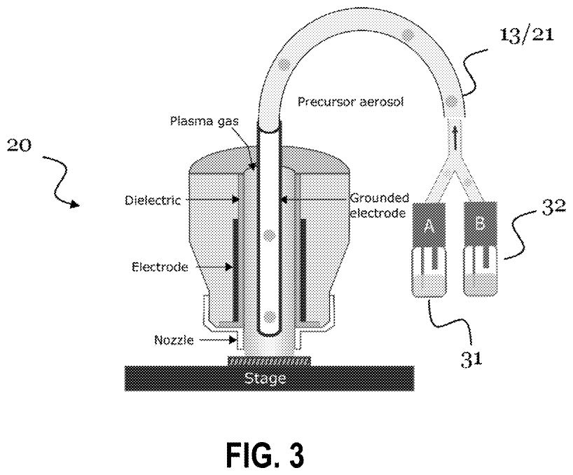

FIG. 2 and FIG. 3 illustrate example devices 20 for codepositing the biomolecule 12 and the organic precursor 13 using APPD. The APPD devices 20 may generate a plasma in a chamber where a high-voltage electrode ionizes a flowing gas, while a grounded electrode stabilizes the discharge. The grounded electrode may be a hollow electrode. A dielectric barrier prevents arcing, ensuring a uniform plasma that exits through a nozzle and directs the biomolecule 12 and organic precursor 13 onto the surface of a substrate or biosensor 10 placed on a stage. This device setup enables precise codepositing at atmospheric pressure without requiring a vacuum system.

The organic precursor 13 may be injected as a vapor or as an aerosol into the plasma afterglow, while the biomolecule 12 and a mediator may be injected as an aerosol. The biomolecule 12 can be either dissolved in the organic precursor 13, or can be injected as a separate aerosol. The organic precursor 13 and the biomolecule 12 (i.e., many molecules of the biomolecule 12) may then be copolymerized by the plasma afterglow, or may be immobilized in a different way, e.g., electrostatically or by steric embedding. This provides stability to the biomolecule 12, preventing it from escaping from the film on the surface 11. Alternatively, the biomolecules are trapped in the film by the polymerized organic precursor matrix.

The difference between the setups of the device 20 shown FIG. 2 and FIG. 3, respectively, is that with the device 20 of FIG. 2 the biomolecule 12 and the organic precursor 13 are injected from a common container 22, while with the device 20 of FIG. 3 the biomolecule 12 and the organic precursor 13 are injected from two separate containers 31 and 32 into the plasma afterglow.

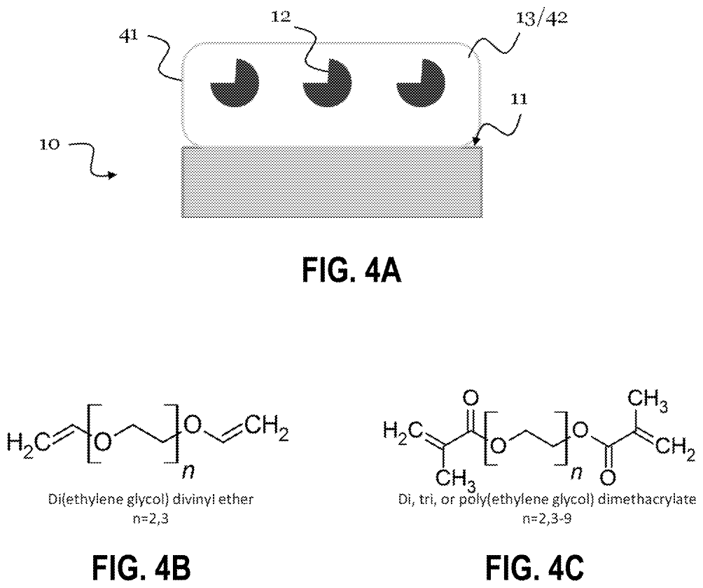

As shown in FIG. 4A, the codeposition of the biomolecule 12 and the organic precursor 13 using APPD leads to a surface 11, on which the biomolecule 12 is functionalized. A film 41 comprising the biomolecule 12 and the organic precursor 13 may be formed on the surface 11. The film 41 may comprise a matrix 42 formed by (derived from) the ethylene oxide containing organic precursor 13, and comprises the (molecules of the) biomolecule 12 dispersed in the matrix 42. Thereby, the molecules of the biomolecule 12 may also be distributed on the surface of the matrix 42.

In an example embodiment, di(ethylene glycol) divinyl ether, which is shown in FIG. 4B, as the organic precursor 13 and glucose oxidase as the biomolecule 12 were codeposited. Another example of the organic precursor 13 is shown in FIG. 4C.

In example embodiments, the organic precursor 13 and the biomolecule 12 may be injected as separate aerosols into the plasma afterglow. Argon may be used as a plasma gas, as it limits heating of the surface, and the air content in the environment may be controlled during deposition of the film 41. The resulting cross-linked polymer film 41 (schematically illustrated in FIG. 4A) is stable in an aqueous environment, has antifouling properties, and contains glucose oxidase with retention of its enzymatic activity, as the film 41 is functional for the electrochemical detection of glucose. The obtained film 41 is ideally suited for bio sensing, and meets the following three criteria:

The stability of the film 41 in the aqueous environment is achieved thanks to the polymerizable groups of the organic precursor 13, which are activated by the plasma of the APPD. The substrate surface providing the surface 11 can also be activated by the plasma, improving the adhesion.

The film 41 is further antifouling thanks to the ethylene oxide contained in the organic precursor 13. The organic precursor 13 particularly prevents fouling by proteins or cells. Organic precursors 13 with high ethylene oxide content are beneficial, which is made possible by injecting the organic precursor 13 as an aerosol.

The biomolecules 12 in the film 41 are still active, because the biomolecule 12 only contacts the plasma afterglow, reducing the fragmentation and/or denaturation of the biomolecule 12. A plasma close to room temperature and with a low energy can be used for cross-linking the film thanks to the polymerizable groups.

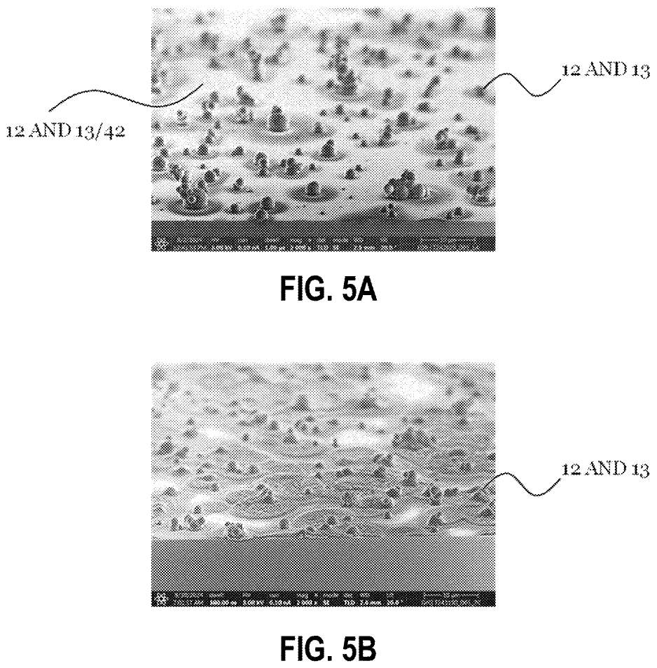

FIG. 5 shows images of a functionalized surface comprising a matrix of an organic precursor 13 and a biomolecule 12. Thereby, FIG. 5A is an image of an organic precursor matrix obtained without the use of a water aerosol, and FIG. 5B is an image of an organic precursor matrix 13 and a biomolecule 12, with addition of a water aerosol. Both images are the result of codepositing with APPD from a single container 22 (as shown in FIG. 2). The biomolecule 12 and the organic precursor 13 are mixed in both cases. Only in FIG. 5B they are more homogeneously distributed.

### EXAMPLES

### Material and Methods

P-type silicon wafers with (100) crystal orientation were pre-cleaned using a two-step cleaning sequence with an O₃ based step for oxidation and diluted HF for subsequent oxide removal. A clean chemical oxide was regrown using ozonated deionized water (DIW) in the final rinsing step. The thickness of the oxide is around 1 nm, as was verified using ellipsometry and is not affected by subsequent air exposure. The wafers were then cleaved into 3 cm×3 cm coupons. Particles generated during cleaving were removed by subsequent sonication in acetone and isopropanol for 10 minutes. Immediately before plasma deposition, the substrates received an additional 15 min UV/O₃ clean to remove any airborne organic contamination.

All plasma depositions were performed using a DBD plasma jet (Molecular Plasma Group (MPG), Luxembourg) DBD plasma jet. The DBD plasma jet powered electrode was fed with a high-voltage up to 15 kV peak-to-peak, at a frequency in the 40-50 kHz range. The nominal power output was from 90 to 150 W. A fixed flow rate of 80 standard liters per minute (slm) Argon (Alphagaz, Airliquide) was fed to the system acting as discharge gas.

The organic precursor and the biomolecule were provided inside the hollow inner electrode in the form of an aerosol generated by an atomizer.

In case of water co-injection, a water aerosol was generated by a second atomizer and the two aerol-flows were merged in a Y-splitter. Plasma deposition was performed across the entire sample surface area by scanning the plasmajet in a serpentine pattern.

### Example 1

Poly(ethylene glycol) dimethacrylate (PEGDMA) with a molecular mass of 750 g/mol was used as the organic precursor. Glucose Oxidase (GOx) from Aspergillus niger type X-S with 100,000-250,000 units/g solid was used as the biomolecule.

A precursor aerosol is generated from a solution of 25 mg/mL GOx (Glucose Oxidase) in water with 10 vol % PEGDMA. The aerosol is transported to the plasma jet and combined with the plasma afterglow in the nozzle to obtain a deposited GOx/PEO film.

The principle for a first generation amperometric biosensor with a Pt working electrode is well established, with FAD the flavin adenine dinucleotide, the cofactor of GOx that is reduced:

β-D-Glucose+GOx-F AD⇒GOx-F ADH₂+d-D-gluconolactone

GOx-F ADH₂+O₂⇒GOx-F AD+H₂O₂

The Pt working electrode is covered with the AAPD films, and oxidation of glucose by the GOx in the film generates H₂O₂, which is detected at the Pt electrode at a potential of 0.6 V vs Ag/AgCl:

H₂O₂⇒2e⁻+O₂+2H⁺

Dose-response curves were obtained in static phosphate buffered saline (PBS), and were fitted with the Michaelis Menten equation:

$$J = A + {J}_{m ax} \frac{c}{{K}_{M} + c}$$

with J the current density, c the glucose concentration, Jₘₐₓ the current corresponding to the maximum reaction rate, K_{M} the Michaelis Menten constant and A an offset to account for the non-zero background.

Electrochemical experiments were performed using an Autolab potentiostat from Metrohm. The three-electrode cell consisted of a BASi MW-1033 coiled platinum counter electrode, a BASi RE-5B silver-chloride reference electrode and a working electrode functionalized with the plasma deposited layer. Except where explicitly stated, experiments were performed in a stationary solution. Before amperometry, three cyclic voltammetry scans between 0.7 V and −0.1 V at a scan rate of 0.1 V/s were performed. Subsequently, a potential of 0.6 V was applied for 5 minutes to obtain a stable background signal. The solution was then titrated with glucose by adding different volumes of a 1 M stock solution of glucose in water. After every glucose addition the solution was briefly stirred with a magnetic stirrer. Signals for every concentration were recorded for 80 seconds at a potential of 0.6 V. The reported currents are the plateau values after 80 seconds.

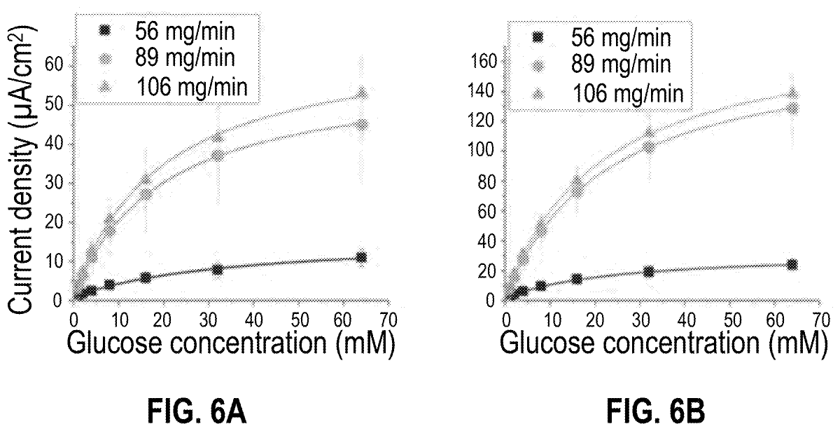

The dose-response curves are depicted in FIG. 6, without (FIG. 6A) and with water (FIG. 6B) coinjection. 56, 89, 106 mg/min refer to the mass flow of the GOx/PEGDMA/H₂O aerosol.

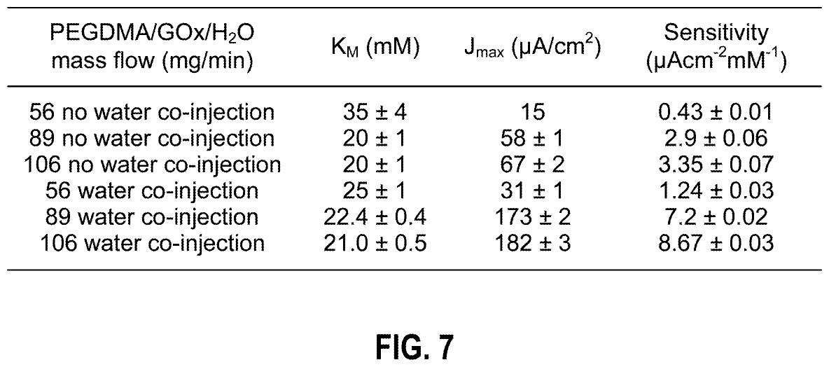

FIG. 7 shows a table with the apparent K_{M}, Jₘₐₓ, and sensitivity for the films deposited at different precursor mass flows and with and without water co-injection. The sensitivity was calculated as Jₘₐₓ/K_{M}, the initial slope of the fitted curve.

For all conditions the signal clearly differs from the background, indicating that the enzymes retained at least part of their catalytic activity after AAPD.

For all precursor mass flows, co-injecting water during AAPD consistently improves the sensitivity of the sensor.

The sensitivity of the GOx/PEO films is in the range with typical sensitivities found in literature for first generation glucose biosensors where glucose oxidase was entrapped in different matrices demonstrating that AAPD can be successfully used for the deposition of a GOx sensing layer.

### Example 2

A deposited GOx/PEO film was prepared according to Example 1.

To assess the performance of the sensing layer in biologically relevant media, a dose-response curve was made in cell culture medium supplemented with 10 vol % fetal bovine serum (FBS).

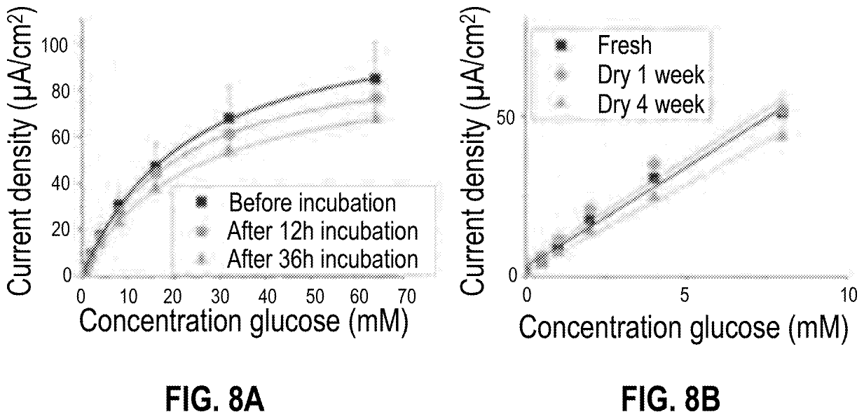

Dose-response curves were recorded in PBS immediately after incubation of the sensor in cell culture medium with 10% FBS for 12 h and 36 h (FIG. 8A). After 12 h, the sensitivity of the sensor is similar to before incubation in medium. After 36 h incubation a sensitivity of 3.8 μAcm⁻² mM⁻¹ is still retained, demonstrating that fouling does not limit the sensitivity of the GOx/PEO films. Finally, the shelf life of the sensor was evaluated.

FIG. 8B shows dose-response curve for glucose in PBS for a film deposited at 106 mg/min mass flow with water co-injection flow after storing the film in a dry environment for 1 and 4 weeks, according to example embodiments. Curves were fitted linearly between 0.5 mM and 8 mM. Error bars represent standard deviations on three samples. As shown in FIG. 8B, the sensor retains full sensitivity after one week dry storage at room temperature, and ≈87% of its initial sensitivity after four weeks. This demonstrates the biocompatibility of the PEO matrix and its effectiveness in preserving the enzyme activity.

### Example 3

A deposited GOx/PEO film was prepared according to Example 1.

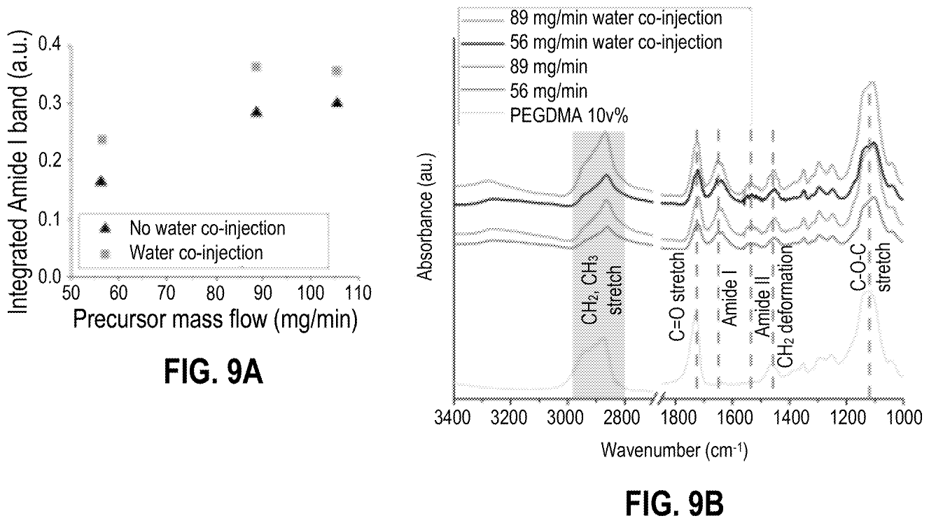

The film composition was analyzed by FT-IR spectroscopy (FIG. 9B).

The FT-IR spectra confirm the presence of deposited GOx, indicated by the appearance of characteristic amide I and amide II bands (at 1650-1690 cm⁻¹ and 1515-1570 cm⁻¹ respectively, FIG. 9B). These bands are absent in spectra of films deposited from PEGDMA without GOx. FIG. 9A shows the integrated amide I band intensity, serving as a quantitative measure of the amount of deposited GOx. For the same precursor mass flow, co-injecting water increases the amount of deposited GOx. This effect can be attributed to improved aerosol collection efficiency: co-injecting water leads to larger droplets reaching the substrate. Larger droplets are collected more efficiently on the substrate compared to smaller droplets, increasing the material balance.

Bands associated with PEO are also seen in the FT-IR spectra (FIG. 9B): the band at 1130 cm-1 representing stretching vibration of the ether bonds, a broad band containing the symmetric CH2 stretch (2870-2840 cm-1) and the asymmetric CH2 stretch (2940-2915 cm-1), overlapping with the symmetric CH3 stretch (2940-2880 cm-1) and the asymmetric CH3 stretch (2975-2950 cm-1), and the CH2 deformation vibration (1450 cm-1). This suggests that the CH2-CH2-O units from the PEGDMA precursor are at least partially retained in the deposited films, and that the films are biocomposites of a PEO-like matrix and GOx. FIG. 9B also shows a reference spectrum of a film deposited from 10 vol % PEGDMA. No normalization was performed.

In sum, this disclosure provides a solution for functionalizing a surface with a biomolecule, wherein the use of APPD enables a one-step process, and the selection of the organic precursor 13 leads to a stable layer with strong cross-linking and antifouling properties.

In the claims as well as in the description of this disclosure, the word “comprising” does not exclude other elements or steps and the indefinite article “a” or “an” does not exclude a plurality. A single element may fulfill the functions of several entities or items recited in the claims. The mere fact that certain measures are recited in the mutual different dependent claims does not indicate that a combination of these measures cannot be used in an example implementation.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A method for functionalizing a surface of a biosensor with a biomolecule comprising:
codepositing the biomolecule and an organic precursor onto the surface using atmospheric pressure plasma deposition (APPD),
wherein the organic precursor comprises a polymerizable group and an ethylene oxide functional group.

2. The method of claim 1, wherein the polymerizable group of the organic precursor comprises a vinyl, an acrylate, a methacrylate, an epoxy, or a silane.

3. The method of claim 1, wherein the organic precursor comprises at least two polymerizable groups.

4. The method of claim 1, wherein the organic precursor further comprises a spacer, and wherein the spacer comprises an alkane, an ether, a ketone, an ester, or an amide.

5. The method of claim 1, wherein the biomolecule comprises one of an enzyme, an antibody, a nanobody, an aptamer, DNA, RNA, or a peptide.

6. The method of claim 1, wherein the organic precursor comprises di(ethylene glycol) divinyl ether or poly(ethylene glycol) dimethacrylate.

7. The method of claim 1, wherein the organic precursor comprises ethylene glycol dimethacrylate (EGDMA), di(ethylene glycol) dimethacrylate (2EGDMA), tri(ethylene glycol) dimethacrylate (3EGDMA), ethylene glycol divinyl ether, tri(ethylene glycol) divinyl ether, or polyethylene glycol divinyl ether.

8. The method of claim 1, wherein the biomolecule comprises glucose oxidase.

9. The method of claim 1,
wherein codepositing the biomolecule and the organic precursor onto the surface using APPD comprises using a plasma afterglow during the APPD; or
wherein subsequent to codepositing the biomolecule and the organic precursor, the method comprises treating the biomolecule and the organic precursor with the plasma afterglow.

10. The method of claim 9, wherein codepositing the biomolecule and the organic precursor further comprises:
injecting the organic precursor as vapor or an aerosol into the plasma afterglow; and
injecting the biomolecule as an aerosol into the plasma afterglow.

11. The method of claim 9,
wherein the biomolecule and the organic precursor are dissolved in water, and
wherein codepositing the biomolecule and the organic precursor further comprises injecting the water comprising the dissolved organic precursor and biomolecule into the plasma afterglow.

12. The method of claim 9, wherein codepositing the biomolecule and the organic precursor further comprises injecting the biomolecule and the organic precursor from a common container into the plasma afterglow.

13. The method of claim 9, wherein codepositing the biomolecule and the organic precursor further comprises injecting the biomolecule and the organic precursor from separate containers into the plasma afterglow.

14. The method of claim 9, wherein codepositing the biomolecule and the organic precursor further comprises injecting a water aerosol together with the organic precursor and the biomolecule into the plasma afterglow.

15. The method of claim 1, wherein argon, helium, or nitrogen is used as plasma gas for the APPD.

16. The method of claim 1, wherein codepositing the organic precursor and the biomolecule forms a film on the surface.

17. The method of claim 1, wherein codepositing of the organic precursor and the biomolecule is performed with an inert gas flux.

18. A biosensor comprising:
a surface on which a biomolecule is immobilized,
wherein a film comprising the biomolecule and an organic precursor is formed on the surface, and
wherein the organic precursor comprises a polymerizable group and an ethylene oxide functional group.

19. The biosensor of claim 18, wherein the film comprises:
a matrix formed by the ethylene oxide containing organic precursor; and
the biomolecule dispersed in the matrix.

20. The biosensor of claim 18, wherein the biomolecule is distributed in the film or on top of the film.
